(19) United States
(12) Patent Application Publication
(10) Pub. No.: US 2016/0115536 A1
(43) Pub. Date: Apr. 28, 2016

(54) METHODS FOR ESTIMATING THE SIZE OF DISEASE-ASSOCIATED POLYNUCLEOTIDE REPEAT EXPANSIONS IN GENES

(71) Applicant: MEDICAL RESEARCH COUNCIL, Swindon (GB)

(72) Inventors: Simon Mead, London (GB); Mark Poulter, London (GB); Jonathan Beck, London (GB)

(21) Appl. No.: 14/762,550

(22) PCT Filed: Jan. 20, 2014
(86) PCT No.: PCT/GB2014/050148

§ 371 Date: Jul. 22, 2015

(30) Foreign Application Priority Data
Jan. 23, 2013 (GB) 1301164.8

(51) Int. Cl. C12Q 1/68

(52) U.S. Cl. CPC C12Q 1/6883; C12Q 2600/158

(57) ABSTRACT

Methods for estimating the size of disease-associated polynucleotide repeat expansions in genes are disclosed which use restriction enzymes that do not cut within a repeat expansion and which are frequent cutting restriction enzymes that cut genomic DNA outside of the expansion into fragments of a size below the threshold capable of detection. A hybridisation probe that can bind to multiple sites within the expansion is then used to estimate its length and to correlate that to the diagnosis or prognosis of disease.

### FIELD OF THE INVENTION

The present invention relates to methods for estimating the size of disease-associated polynucleotide repeat expansions in genes, and in particular for estimating repeat expansions of large size.

### BACKGROUND OF THE INVENTION

It is known in the art that some diseases or conditions, in particular some neurodegenerative disorders, are characterised by mutations in which polynucleotide repeat expansions accumulate within a gene sequence. Often these repeat expansions are present in the genomes of healthy individuals and the point at which these mutations become pathogenic is often dependent on the length of the repeat expansion. Estimating the pathogenic size range, mutation mechanisms, the feasibility and accuracy of diagnostic testing and genotype-phenotype correlations is therefore of considerable interest in the diagnosis and prognosis of these conditions, as well as in the scientific study of their causes.

By way of example, large expansions of a non-coding GGGGCC repeat in C9orf72 have recently been identified as an important cause of frontotemporal dementia (FTD), motor neuron disease (MND) and the combined syndrome (FTD-MND; Renton et al., 2011; Dejesus-Hernandez et al., 2012). The finding is remarkable because of the high mutation prevalence in these disease syndromes and because the nature of the mutation implies a distinct mechanism of neurodegeneration. However, the discovery of the causal mutation and its further investigation has been hampered by the extremely large size of many expansions which prevent amplification of the entire expansion using conventional PCR-based methods. Thus, DNA from the expansion allele can be amplified using a PCR with primers complementary to the repeat (repeat-primed or rpPCR), however this method cannot size accurately beyond around 30 repeats (Renton et al., 2011), whereas repeats are often pathogenic only when they reach significantly higher repeat numbers.

In the absence of a workable PCR based method for estimating the size of an expanded repeat, those working in this field have turned to conventional Southern hybridisation techniques. When used to analyse an expanded repeat, Southern blotting involves the digestion of gDNA with a restriction endonuclease, resolving the fragments by electrophoresis and the use a probe that identifies single copy sequence adjacent to the expanded repeat and within the same restriction fragment. By identifying and detecting this fragment, the size difference caused by variation in the repeat number can be detected. A signal produced from such a probe under suitably stringent conditions will originate only from its complementary sequence and is therefore highly specific.

Unfortunately this conventional method is often difficult to perform, in particular where there is instability in the repeat length in different cells of mutation carrying individuals. As a result, the fragments containing the expansion do not migrate to one point in the gel during electrophoresis, but instead are spread over a wide molecular weight range. This in effect results in a dilution of the signal for a given amount of gDNA blotted as the signal becomes spread over a wider area of the blot significantly reducing sensitivity.

U.S. Pat. No. 6,150,091 describes a method relating to the diagnosis of Friedreich's ataxia (FRDA), in which the approximate number of repeats of the trinucleotide “GAA” in an intron of X25 is determined. This method uses standard Southern blotting of the region of interest and is employed to distinguish between trinucleotide sequence repeat tracts of 1-120 and 120+ repeats, up to a total size of 2700 base pairs. U.S. Pat. No. 6,524,791 relates to the detection of spinocerebellar ataxia type 8 (SCA8)-associated trinucleotide expansions using PCR and standard Southern blotting based methods, the latter being able to detect sequence repeat expansions of up to ˜700 repeats (˜2100 base pairs).

Accordingly, there is at present an unmet need for a reliable and sensitive technique for estimating the size of repeat expansions, and in particular large repeat expansions that cannot be determined using prior art techniques, to help in the study of conditions characterised by the occurrence of these mutations and for use in the diagnosis and prognosis of patients.

### SUMMARY OF THE INVENTION

Broadly, the present invention is based on the development of a protocol for estimating the size of disease-associated polynucleotide repeat expansions in genes, and in particular for estimating the size of large repeat expansions, that overcomes many of the disadvantages associated with conventional Southern hybridisation and PCR techniques. In the present invention, this is achieved through the design of a hybridisation probe and the preparation of the nucleic acid sample used in the hybridisation reaction from genomic DNA.

In contrast to the prior art, the hybridisation probe used in the methods of the present invention is generally not a single copy of a target sequence and therefore would not normally be used in Southern hybridisation because of the risk that it will hybridise at several or many positions within the genome, thereby resulting in a number of signals which may not be easily distinguishable from each other. For example, in the examples set out below, the hybridisation protocol uses an oligonucleotide repeat probe (e.g. (GGGGCC)₅; SEQ ID NO: 1) which targets multiple sites within the expansion (e.g. GGGGCC) and will hybridise potentially to other sites within the genome because of its lack of complexity. However, when the design of the hybridisation probe is combined with genomic DNA (gDNA) digested with one or more frequently cutting restriction endonucleases (such as AluI and DdeI) having restriction sites that closely flank the expanded repeat region, the method is specific for the repeat expansion because the restriction enzymes shatter the gDNA outside of the repeat to a modal size (e.g. 200-300 bp) which is much smaller a modal size than necessary for genomic Southern hybridisation protocols. This highly fragmented gDNA allows the hybridisation probe to have both hybridisation sensitivity and specificity for the repeat expansion because the probability of another repeat containing a fragment of similar size to the disease causing expansion in the gene in question is very low. Specificity may also be supported when interpretation of Southern blot data made together with results from rpPCR amplification which utilises primers complimentary to unique flanking sequence.

Moreover, the size of the fragment containing the repeat expansion enables the signal it generates from hybridisation to the probe to be clearly separated from any other signals generated elsewhere in the genome most of which are lost either because digested fragments are so small that they run off the end of the gel during electrophoresis or they are unable to blot efficiently. Fortunately, the hybridisation probe does detect a smaller target in both affected and unaffected individuals so there is always an internal control signal to monitor the efficiency of the method. This mimics the usefulness of the normal allele signal when using a single copy probe. Sensitivity is achieved because the hybridisation probe although small as compared with most single copy probes has multiple hybridisation sites within the expansion. The combination of a double digest with frequent cutting endonucleases and a probe that has multiple targets within the expanded repeat results in significantly increased sensitivity to a conventional Southern blotting, whilst matching the specificity of a single copy probe. The methods of the present invention are therefore capable of being used for estimating the size of massive repeat expansions that are outside of the limits of other techniques, such rpPCR or conventional Southern blotting.

Accordingly, in a first aspect, the present invention provides a method of estimating the size of a disease-associated polynucleotide repeat expansion in a gene, the method comprising:
(a) contacting the sample of genomic DNA from an individual with one or more restriction enzymes, wherein the restriction enzymes have restriction sites flanking the region of genomic DNA containing the polynucleotide repeat expansion and are capable of cutting the genomic DNA outside of the fragment containing the polynucleotide repeat expansion into a plurality of DNA fragments;
(b) optionally separating the nucleic acid fragment containing the polynucleotide repeat expansion from the plurality of DNA fragments;
(c) contacting the nucleic acid fragment containing the polynucleotide repeat expansion with a hybridisation probe capable of targeting multiple sites within the polynucleotide repeat expansion; and
(d) detecting the hybridisation of the hybridisation probe to the polynucleotide repeat expansion to estimate the size of the disease-associated polynucleotide repeat expansion.

Preferably, the restriction enzymes used to cut the sample of genomic DNA do not cut within the region containing the polynucleotide repeat expansion. This maintains the integrity of the target polynucleotide repeat sequence and allows estimation of its size.

The restriction enzymes used to cut the sample of genomic DNA generally produce DNA fragments of a modal size below the size of the repeat expansion, i.e. below a repeat expansion length that is capable of being detected by the method of the invention, allowing polynucleotide repeat sequences to be detected by resolution of fragmented genomic DNA samples by size.

Preferably, the restriction enzymes used to cut the sample of genomic DNA produce DNA fragments with a modal size no greater than 500 base pairs in length. More preferably, the DNA fragments have a modal size no greater than 400 base pairs, or more preferably still 300 base-pairs. This allows for detection of polynucleotide repeat sequences of a size above a modal size of 500, 400 and 300 base-pairs, respectively.

Generally, the method of the invention comprises contacting the sample of genomic DNA with more than one restriction enzyme. The use of more than one restriction enzyme facilitates fragmentation of genomic DNA to a modal size appropriate for the method of the invention. For example, the restriction enzymes used in the method of the invention may be AluI and DdeI.

Preferably, the restriction sites for the restriction enzymes are within a distance (in base pairs) less than the modal size of the fragmented DNA from the 3′ and/or 5′ ends of the polynucleotide repeat sequence, allowing for accurate estimation and/or determination of the size of the polynucleotide repeat sequence.

Generally, the method of the invention comprises one or more hybridisation probes for the detection of the presence or size of a polynucleotide repeat expansion.

Preferably, the hybridisation probe of the method of the invention comprises a multimeric sequence capable of hybridising to the polynucleotide repeat expansion, increasing specificity for the target polynucleotide repeat sequences.

Preferably, the hybridisation probe comprises one or more repeats of a sequence capable of hybridising to a sequence comprising at least one tandem repeat of a polynucleotide sequence. Preferably the polynucleotide sequence tandem repeat is comprised in a polynucleotide repeat expansion. Probes may comprise repeats of the polynucleotide sequence or a complement thereof. More preferably, the probe comprises 2, 3, 4, 5, 6, 7, 8, 9 or 10 repeats of a sequence capable of hybridising to a polynucleotide repeat expansion.

Generally, the hybridisation probe comprises a label for the detection of hybridisation to a polynucleotide repeat region. For example, the label may a fluorescent, chemiluminescent, chromogenic, enzymatic, radioactive or hapten label. Preferably, the label is a hapten, more preferably the hapten is digoxigenin (DIG). Such labels facilitate detection of hybridisation of the probe to a polynucleotide repeat sequence and thus detection. Probes may be labelled at multiple sites to amplify signal from the probe. Hapten labels have the advantage of an indirect detection step, further amplifying the signal from the hybridisation probe and thereby increasing the sensitivity of the method.

Preferably, the polynucleotide repeat expansion detected by the method of the invention comprises 100 repeats or more. More preferably, the repeat expansion may comprise 50 or 20 repeats or more. The method of the invention is versatile and is capable of detecting expansions across a range of sizes.

Generally, the total size of the repeat expansion for detection by the method of the invention is at least about 1650 base pairs in length. The method is therefore capable of detecting expansions beyond the range detectable with rpPCR methods and/or conventional Southern blot methods.

The size of polynucleotide repeat expansions determined by the method of the invention may be estimated by reference to one or more DNA fragments of a known size.

The size of the polynucleotide repeat expansion detected by the method of the present invention may be variable in a sample taken from an individual.

Preferably, the method of the invention comprises a step of determining the range of variation in the size of polynucleotide repeat expansions in a sample from an individual. This is not possible using less-sensitive single-copy probes of conventional Southern blotting methods.

Generally, the method of the invention does not comprise a step of amplifying the genomic DNA sample obtained from the individual, and is capable of estimating the size of a repeat expansion in a DNA sample of 5 μg or less, or even 3 μg or less. The method therefore requires smaller starting DNA sample sizes compared with conventional Southern blotting methods (˜5-10 ug) for the detection of polynucleotide repeat sequence expansions.

Generally, the method of the invention comprises a step of separating nucleic acid fragments containing polynucleotide repeat expansions from the plurality of smaller DNA fragments generated by restriction digestion of the DNA sample, allowing polynucleotide repeat sequences to be easily distinguished from smaller, non-repeat sequence DNA fragments.

Preferably, separation of nucleic acid fragments containing polynucleotide repeat expansions from the plurality of smaller DNA fragments generated by restriction digestion of the DNA sample is achieved by electrophoresis.

Generally, the method of the present invention can be used to inform the diagnosis of, predisposition to, clinical phenotype and/or prognosis of, and/or response to treatment for the disease associated with the expansion of polynucleotide repeats. The method of the invention can thus inform counselling and therapeutic decisions.

Preferably, the disease associated with the presence or size of a polynucleotide repeat expansion can be diagnosed using the method of the invention.

Accordingly, the method of the present invention may comprise an additional step of:
correlating the estimated size of the polynucleotide repeat expansion with the range of sizes considered to be non-pathogenic or pathogenic for the disease, wherein an estimated size within the range considered to be pathogenic is indicative of disease.

Preferably, predisposition of the offspring of an individual to the disease associated with the presence or size of a polynucleotide repeat expansion can be determined using the method of the invention.

Accordingly, the method of the present invention may comprise an additional step of:
correlating the estimated size of the polynucleotide repeat expansion with the range of sizes considered to be non-pathogenic or pathogenic for the disease, wherein an estimated size between these two ranges or in the upper 10% of expansion sizes in the non-pathogenic range is indicative of a predisposition of offspring of the individual to the disease.

Preferably, the age of onset of the disease associated with the presence or size of the polynucleotide repeat expansion can be estimated using the method of the invention.

Accordingly, the method of the present invention may comprise an additional step of:
correlating the estimated size of the polynucleotide repeat expansion with the range of sizes associated with a particular age of onset for the disease, wherein larger repeat expansion sizes within the pathogenic range is indicative of an earlier age of onset for the disease.

Preferably, clinical phenotype for the disease associated with the presence or size of the polynucleotide repeat expansion can be informed using the method of the invention.

Accordingly, the method of the present invention may comprise an additional step of:
correlating the estimated size of the polynucleotide repeat expansion with the range of sizes associated with a particular disease clinical phenotype.

Preferably, prognosis for the disease associated with the presence or size of the polynucleotide repeat expansion can be informed using the method of the invention.

Accordingly, the method of the present invention may comprise an additional step of:
correlating the estimated size of the polynucleotide repeat expansion with the range of sizes associated with a particular disease prognosis, wherein larger repeat expansion sizes within the pathogenic range is indicative of a poorer disease prognosis.

Preferably, response to treatment for a disease associated with the presence or size of the polynucleotide repeat expansion can be estimated using the method of the invention.

Accordingly, the method of the present invention may comprise an additional step of:
correlating the estimated size of the polynucleotide repeat expansion with the range of sizes associated with a particular response to treatment for a disease.

The method of the invention may be performed on a sample from an individual in which a polynucleotide repeat expansion has already been identified, by rpPCR, PCR, DNA sequencing or conventional Southern blotting techniques, preferably by rpPCR. This will support analysis of polynucleotide repeat sequence expansions using the method of the invention.

The disease associated with the presence or size of polynucleotide repeat expansion may be a neurological disease. Preferably, the neurological disease is a neurodegenerative disease. Examples of diseases associated with presence or size of polynucleotide repeat expansions include frontotemporal dementia (FTD), amyotrophic lateral sclerosis (ALS), motor neuron disease (MND), Alzheimer's disease (AD), Huntington's disease (HD), Friedreich's ataxia (FRDA), X-linked spinal and bulbar muscular atrophy (SBMA), fragile X syndrome (FRAXA), fragile X associated tremor/ataxia syndrome (FXTAS), fragile XE mental retardation (FRAXE), myotonic dystrophy (DM), spinocerebellar ataxias (SCRs), corticobasal syndrome (CBS), ataxic syndrome and dentatorubal-pallidoluysian atrophy (DRPLA). The method of the invention is therefore appropriate for use in the analysis of polynucleotide repeat sequence expansions associated with a wide range of diseases.

Accordingly, the present invention provides a method for detecting a polynucleotide repeat expansion associated with a disorder listed in Table 1, using a hybridisation probe with a multimeric sequence corresponding to a polynucleotide repeat sequence listed in Table 1 or a complement thereof.

Generally, the present invention provides a method for detecting the presence or size of a GGGGCC polynucleotide repeat expansion in the C9orf72 gene using a hybridisation probe comprising the sequence (GGGGCC)n, where n is between 2 and 10 (SEQ ID NO: 2). Equally, the hybridisation probe may have the sequence (CCCCGG)n (SEQ ID NO: 3), which is capable of hybridising to the complementary DNA strand at GGGGCC repeats.

In a further aspect, the present invention provides a kit for estimating the size of a disease-associated polynucleotide repeat expansion in a gene, the kit comprising:
one or more restriction enzymes, wherein the restriction enzymes have restriction sites flanking the region of genomic DNA containing the polynucleotide repeat expansion and which are capable of cutting the genomic DNA outside of the polynucleotide repeat expansion into a plurality of small DNA fragments;
a hybridisation probe capable of targeting multiple sites within the polynucleotide repeat expansion; and
wherein detecting the hybridisation of the hybridisation probe to the polynucleotide repeat expansion enables the size of the disease-associated polynucleotide repeat expansion to be estimated.

Embodiments of the present invention will now be described by way of example and not limitation with reference to the accompanying figures. However various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### BRIEF DESCRIPTION OF THE FIGURES

FIG. 1. Histogram showing frequency of C9orf72 repeat sizes from 1 to 32 in 1958 Birth Cohort (58BC) 58BC UK healthy controls and the entire CEPH sample collection. rs3849942G associated repeats are shown in green and rs3849942G (“risk” haplotype marker) are shown in red. Phase of genotypes with repeat size was calculated for the CEPH individuals and frequencies then applied to 58BC data.

FIG. 2. Schematic of Southern blot data for 57 cases and 11 controls showing C9orf72 repeats sizes across 7 cohorts. Individual blot data is represented by a coloured bar, with modes indicated with similar coloured dots and the midpoint of size with a vertical black bar. Ages of onset where available are given in years at the right hand end of individual bars. DNA was extracted from tissues as shown on the left. In 3 healthy controls data is shown for lymphocyte cell line DNA (LCL) as well as peripheral blood DNA, pairs are shown with in parentheses. *Unusual MND case with doublet of bands of relatively low size; **single 58BC individual with large repeat size from LCL with diagnosis of MND.

FIG. 3. Southern blot showing C9orf72 repeat expansions in 8 cases and 1 ECACC and 2 58BC healthy controls demonstrating typical banding patterns and lower size in lymphocyte cell line DNA than DNA from blood. Control DNA without an expansion is also shown. Case 1 and Case2 show Southern blotting of DNA from 3 different brain regions. *additional bands of probable G4C2 containing short tandem repeat genome motif unrelated to C9orf72.

FIG. 4. Southern blot showing data from 3 58BC healthy controls with C9ORF expansions for both peripheral blood DNA and lymphocyte cell line (LCL) DNA. Typical LCL banding patterns can be seen and may represent pauciclonality of cell line DNA. The size of repeats associated with cell line DNA is smaller than repeats seen in peripheral blood DNA which is similar in size to case DNA. * additional bands of probable G4C2 containing short tandem repeat genome motif unrelated to C9orf72.

### DETAILED DESCRIPTION

The present invention is based on work that involved C9orf72, a major new disease gene in frontotemporal dementia (FTD) and motor neuron disease (MND). Understanding of disease mechanisms and a method for clinical diagnostic genotyping has been hindered because of the difficulty in estimating the hexanucleotide repeat expansion size.

In this work, 10553 patient and controls were screened using repeat primed PCR (rpPCR), and a developed a new Southern blot protocol to estimate expansion size in mutation carriers using 68 blood, brain and cell line samples.

A total of 96 rpPCR expansions were found: 28/375 (7.5%) in FTD, 29/360 (8.1%) MND, 11/904 (1.2%) and 7/421 (1.7%) in samples referred for Alzheimer's disease (AD) and Huntington's disease gene testing (HD-like) respectively, 10/914 (1.1%) in samples send for other neurodegenerative diseases, and 12/7579 in UK controls (population prevalence 0.16% (0.08-0.28%)). The estimated case size repeat range using our Southern blot was 800-4400 (smear maxima from 57 cases). Among population controls, the size range was dependent on the DNA source: we detected smaller maxima in DNA from cell lines (800-2600 repeats) than from blood (3700-4400 repeats), however these estimates overlapped those measured in the case series. We found considerable size heterogeneity in single samples, in size patterns, and between brain regions probably due to somatic mutation. Expansion size in blood correlated with age at clinical onset and the presence of a family history, but importantly did not differ between diagnostic groups. Evidence of instability of repeat size in control families, and neighbouring SNP and microsatellite analyses strongly support the risk haplotype hypothesis of mutation origin.

The present inventors realised that this method for estimating the size of large C9orf72 expansions which has potential clinical utility in the diagnosis and/or prognosis of a range of this and other conditions associated with polynucleotide repeat expansions. These are frequent in the healthy population with an estimated 90,000 UK carriers. As the disease may mimic any of several neurodegenerative diseases, expansion-associated syndromes may be more common than currently realised.

### Polynucleotide Repeat Expansions and Associated Diseases

Polynucleotide repeat expansions are associated with the development and/or progression of several diseases, including frontotemporal dementia (FTD), amyotrophic lateral sclerosis (ALS), motor neuron disease (MND), Alzheimer's disease (AD), Huntington's disease (HD), FRDA, X-linked spinal and bulbar muscular atrophy (SBMA), fragile X syndrome (FRAXA), fragile X associated tremor/ataxia syndrome (FXTAS), fragile XE mental retardation (FRAXE), myotonic dystrophy (DM), spinocerebellar ataxias (SCAs), corticobasal syndrome (CBS), ataxic syndrome and dentatorubal-pallidoluysian atrophy (DRPLA).

Polynucleotide repeat sequences arise from the tandem duplication of unstable, 2-6 base pair microsatellite repeat sequences (also known as simple sequence repeats (SSRs) or short tandem repeats (STRs)) that are distributed throughout the genome.

For example, expansions of tandem repeats of the trinucleotides “CAG” and/or “CTG” are associated with a wide range of diseases, and may be found in the coding region of the affected gene, giving rise to so-called poly-glutamine (polyQ) disorders, or may be in untranslated regions (non-polyQ). Poly-Q disorders share certain pathogenic features which are thought to be the result of protein misfolding and aggregation associated with long tracts of glutamine residues in the translated protein.

Expansions of the pentanucleotide “ATTCT” in an intron of SCA10, and of the hexanucleotide repeat “GGCCTG” in NOP56 are further examples of a disease-associated repeat expansions, associated with spinocerebellar ataxias 10 and 36, respectively.

Recently, expansion of the hexanucleotide (GGGGCC) in the first intron of C9orf72 has been associated with the development of FTD, MND, FTD-MND and ALS-FTD.

Expansions of polynucleotide repeat sequences are thought to occur as the result of “slippage” during DNA replication. Slippage occurs when local DNA strand separation occurs in a region of repeats, resulting in the creation of single stranded loops of repetitive sequence that may then be displaced (or “slip”) and result in the addition of further repeats through amplification by DNA polymerases. The stochastic nature of the generation of polynucleotide repeat expansions during DNA replication means that the number of repeats in a given polynucleotide repeat sequence may vary between cells even within a sample from an individual. This makes expansions of variable size difficult to detect by the standard means of detection currently employed.

Disorders caused by the expansion of polynucleotide repeat sequences are associated with anticipation; the tendency for an earlier onset and/or increasingly severe disease symptoms in successive generations. This is thought to be the result of the accumulation of repeats and explains the observation that families with a longer history of, for example, Huntington's disease have earlier onset and poorer prognosis.

Furthermore, pathogenic repeat expansions of certain sizes may be associated with certain clinical phenotypes of disease associated with polynucleotide repeat expansions, and may even be used to inform therapeutic strategy for the treatment of such diseases.

There is therefore significant value in being able to detect the number of repeats and even modest expansions in polynucleotide repeat sequences.

Affected genes have different normal, stable thresholds for the number of repeats, above which disease manifests. Table 1 shows non-pathogenic and pathogenic repeat size expansions for several diseases associated with polynucleotide repeat expansions.

| | | | Normal (non- | | | |
|---|---|---|---|---|---|---|
| | | | pathogenic) | | Pathogenic | |
| | | Expansion | expansion size | | expansion size | |
| Disease | Gene | motif | (SEQ ID NO) | | (SEQ ID NO) | |
| DRPLA | DRPLA | CAG | 6-35 | (4) | 49-88 | (15) |
| HD | HTT | CAG | 10-35 | (4) | >35 | (16) |
| SBMA | AR | CAG | 9-36 | (5) | 38-62 | (17) |
| SCA1 | ATXN1 | CAG | 6-35 | (4) | 49-88 | (15) |
| SCA2 | ATXN2 | CAG | 14-32 | (6) | 33-77 | (18) |
| SCA3 | ATXN3 | CAG | 12-40 | (7) | 55-86 | (19) |
| SCA6 | CACNA1A | CAG | 4-18 | (8) | 21-30 | (20) |
| SCAT | ATXN7 | CAG | 7-17 | (9) | 38-120 | (21) |
| SCA8 | SCA8 | CTG | 16-37 | (10) | 110-250 | (22) |
| SCA12 | SCA12 | NNN at 5′ | 7-28 | | 66-78 | |
| SCA17 | TBP | CAG | 25-42 | (11) | 47-63 | (23) |
| FRAXA | FMR1 | CGG | 6-53 | (12) | >230 | (24) |
| FXTAS | FMR1 | CGG | 6-53 | (12) | 55-200 | (25) |
| FRAXE | FMR2 | GCC | 6-35 | (13) | >200 | (26) |
| FRDA | FXN | GAA | 7-34 | (14) | >100 | (27) |
| DM | DMPK | CTG | 5-37 | (10) | >50 | (28) |

The number of repeats and/or the extent of repeat expansion necessary to result in a pathology therefore depends on the specific polynucleotide repeat sequence, the gene and the associated disease. For example, in Huntington's disease, a (CAG)₁₀₋₃₅ (SEQ ID NO: 4) repeat frequency within the HTT gene result in the production of a protein with normal function, but (CAG)₃₅₊ (SEQ ID NO: 16) is pathogenic. In contrast, (CGG)₆₋₅₃ (SEQ ID NO: 12) in FRM1 is normal, whilst (CGG)₂₃₀₊ (SEQ ID NO: 24) results in FRAXA.

Furthermore, for certain diseases, polynucleotide repeat expansion sizes are known which fall between the range of expansion sizes considered to be pathogenic and the normal, non-pathogenic range. These expansions, as well as expansions in the upper range of non-pathogenic expansion sizes, are associated with an increased risk of disease in the offspring of that individual, due to anticipation.

For example, with reference to Table 1, individuals with a parent with ˜35 CAG repeats in HTT have been shown to be at an increased risk of sporadic HD (i.e., HD in which there is no family history of the disease).

### Methods for Detecting Polynucleotide Repeat Expansions

Expansions of polynucleotide repeats are typically detected using standard methods for analysing a DNA sequence. By way of example and without limitation, such means of analysis include direct sequencing, hybridisation to a probe, restriction fragment length polymorphism (RFLP) analysis, single-stranded conformation polymorphism (SSCP) analysis, heteroduplex analysis, allelic discrimination analysis or melting curve analysis. These assays may be performed in isolation, in combination or sequentially either directly on a DNA sample or on a sample that is first amplified by PCR.

Alternatively, polynucleotide repeat expansions may be inferred from analysis of RNA or protein products of genes in which an expansion has occurred. Those skilled in the art are well able to employ appropriate techniques for detecting polynucleotide repeat expansions in this way.

Detection of the size of an expanded polynucleotide repeat sequence is often complicated by the repetitive nature and large size of expansions, preventing amplification and/or sequencing using standard methods.

Researchers typically employ Southern blotting techniques to overcome these obstacles. Such assays typically comprise gDNA digestion and resolution of fragments by electrophoresis, followed by the use of a probe to detect a single copy sequence adjacent to the expanded repeat within the same fragment.

However, this method is difficult to perform and has low sensitivity. For many polynucleotide repeat expansion-associated disorders there is variation in the extent of repeat expansion due to instability in repeat length in different cells from the same individual. As such, fragments containing expansions do not migrate to one point in the gel during electrophoresis and are instead spread over a wide range of molecular weights. This results in dilution of the signal emitted by the hybridised probe and thereby reduced sensitivity of the assay. Consequently, rare and variable polynucleotide repeat sequence expansions are not reliably detected by this technique and large amounts of DNA (˜5-10 ug) are typically required for analyses.

Researchers have also analysed polynucleotide repeat expansions by repeat primed PCR (rpPCR). This method uses a first oligonucleotide primer complimentary to a region outside of the repeat sequence region and a second primer, complimentary to the junction at the other end of the repeat sequence, which is also able to hybridise randomly across the repeat sequence tract. After initial rounds of PCR, extended second primers can themselves serve as primers for further rounds of amplification. This results in the production of PCR products of varying size, giving a characteristic “stutter” pattern following electrophoresis, which can be analysed to determine the number of repeats. However, this technique has previously been demonstrated to be unable to accurately size expansions beyond ˜30 repeats (Renton et al., 2011).

Accordingly, there is a need for the development of a technique for the sensitive detection and quantification of polynucleotide repeat expansions, suitable for use on modest amounts of genomic DNA.

The method of the present invention is a new method of Southern blotting which overcomes problems associated with the above techniques. It derives its unique sensitivity by (i) specifying the design of a hybridisation probe and (ii) the preparation of the nucleic acid sample used for hybridisation by restriction digestion of genomic DNA.

The sensitivity of the method of the invention is such that polynucleotide sequence repeat expansions are able to be detected in samples of genomic DNA as small as 3 μg and does not require amplification of the DNA sample as with rpPCR. By way of example, in the experimental examples of the invention below, expansions of the (GGGGCC) repeat sequence in C9orf72 were detected using gDNA samples of 3-10 μg.

The sensitivity of the method of the invention further makes it suitable for use in analyses of unstable and variable polynucleotide repeat sequence expansions.

The method is particularly suitable for the analysis of very large polynucleotide sequence repeat expansions. Preferably, the polynucleotide repeat expansion detected by the method of the invention comprises 10, 20, 30, 40 or more preferably 50 repeats or more, to a total repeat sequence expansion of at least 1650 base pairs in length.

### Southern Blotting

Southern blotting typically involves steps of digesting DNA in a sample with restriction enzymes, separation of fragments by electrophoresis, transfer to a membrane, hybridisation of a labelled probe to DNA fragments on the membrane and determination of binding. Under suitably stringent conditions, specific hybridisation of a probe to a test nucleic acid is indicative of the presence of the sequence in the sample.

Those skilled in the art are well able to employ suitable conditions of the desired stringency for selective hybridisation, taking into account factors such as the length of the probe and base composition, temperature and so on. By way of example, stringent conditions include those that: (1) employ low ionic strength and high temperature for washing, for example 0.015 M sodium chloride/0.0015 M sodium citrate/0.1% sodium dodecyl sulfate at 50° C.; (2) employ during hybridisation a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 760 mM sodium chloride, 75 mM sodium citrate at 42° C.; or (3) employ 50% formamide, 5×SSC (0.75 M NaCl, 0.075 M sodium citrate), 50 mM sodium phosphate (pH 6 8), 0.1% sodium pyrophosphate, 5×Denhardt's solution, sonicated salmon sperm DNA (50 mg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

### (i) The Hybridisation Probe

The hybridisation probe used in the method of the present invention contains multiple, tandem copies of a target polynucleotide repeat sequence. By way of example, the probe may contain 2-10 tandem copies of the target repeat sequence; in the examples set out below, the hybridisation protocol uses the oligonucleotide repeat probe (GGGGCC)₅ (SEQ ID NO: 1).

One important feature of preferred hybridisation probes of the invention compared to single-copy hybridisation probes used in traditional methods to detect polynucleotide repeat expansions by Southern blotting described above, is that the hybridisation probe of the invention will hybridise to multiple sites within a polynucleotide repeat sequence. This has the effect of amplifying the signal from a given DNA fragment containing a repeat expansion, so that the method of the invention is more sensitive than conventional methods.

The binding of the probe to DNA may be measured using any of a variety of techniques at the disposal of those skilled in the art. For instance, probes may have a fluorescent, chemiluminescent, chromogenic, enzymatic, radioactive or hapten label. Probes may be labelled at the 3′, 5′ or at both ends of the probe. By way of illustration, in the examples below, the hybridisation probe is labelled at both the 3′ and 5′ ends with the hapten digoxigenin (DIG).

The skilled person is readily able to design such probes, label them and devise suitable conditions for hybridisation reactions and the detection of hybridisation, assisted by textbooks such as Ausubel et al., 1992.

### (ii) Restriction Digestion

The probe of the invention targets multiple sites within a given expansion and will potentially hybridise to other sites within the genome because of its lack of complexity. However, when the design of the hybridisation probe is combined with digestion of genomic DNA with one or more frequently cutting restriction endonucleases having restriction sites that closely flank the expanded repeat region, the method is specific for the repeat expansion.

Restriction enzymes are used to cleave DNA at specific sites by recognising a specific DNA sequence (restriction site). These sequences are typically 4-12 nucleotides long. The small size of restriction sites and the fact that there are only four bases in the genetic code means that multiple restriction sites are often present in a single DNA molecule.

The use of one or more enzymes might be suitable for performing the method of the invention.

The restriction site-enzyme pair(s) selected for use in the method of the present invention have one or more of the following features:
a) The restriction site is not present within the polynucleotide repeat sequence to be analysed; and/or
b) The restriction site(s) flank(s) the polynucleotide repeat sequence to be analysed. Preferably, the restriction site(s) is/are within a distance (in base pairs) less than the modal size of the fragmented DNA from the 3′/5′ end of the polynucleotide repeat sequence; and/or
c) The restriction enzyme(s) cut(s) frequently throughout the genome outside of the polynucleotide repeat sequence.

Typically, the genomic DNA is fragmented to a modal size below the size of the expansion length capable of being detected by the hybridisation probe of the invention. Preferably the restriction enzyme(s) cut(s) genomic DNA into fragments of a modal size no greater than 500, 400 or more preferably 300 base-pairs in length.

Appropriate restriction site/enzymes for use in the method of the invention will depend on the polynucleotide repeat sequence and/or disease being investigated. Those skilled in the art are well able to identify restriction sites/enzymes suitable for use in the method of the invention. Appropriate restriction site/enzymes for use in the method of the invention can be identified, for example, using restriction enzyme site analysis software, such as Webcutter (rna.lundberg.gu.se/cutter2/) or NEBcutter (tools.neb.com/NEBcutter2/).

By way of illustration, the examples below use the restriction enzymes AluI and DdeI, which recognise the restriction sites “AGCT” and “CTNAG”, respectively, to digest genomic DNA outside of the polynucleotide repeat sequence region into fragments of a modal size of ˜200-300 base-pairs.

### EXPERIMENTAL EXAMPLES

### Methods

### DNA Extraction

Genomic DNA was extracted using the Nucleon BACC2 DNA extraction kit (RPN8502) following the supplied protocol. DNA concentrations were determined using a Nanodrop ND-1000 spectrophotometer, and adjusted to 200-250 ng/μl in TE buffer (Dejesus-Hernandez et al., 2012). Concentrations were re-measured and diluted to 20 ng/μl. Some case samples were extracted from brain tissue as previously described (Mahoney et al., 2012).

### Microsatellite Analysis

Microsatellite analysis was performed using ten markers spanning approximately 13.1 Mb of genomic DNA centred around the C9orf72 gene. PCR amplicons were generated using fluorescently end labeled primers at 500 μM for microsatellite markers D9S1814(VIC), D9S976 (FAM), D9S171 (NED), D9S1121 (VIC), D9S169 (FAM), D9S263(HEX), D9S270(FAM), D9S104(FAM), D9S147E(NED) and D9S761(FAM) in MegaMix Royal hot start cocktail (Microzone). Thermal cycling conditions included an initial preheat at 95° C. for 5 minutes, followed by 35 cycles of 95° C. 30″, 58° C. 40″, 72° C. 1′. A loading mix of 1 μl amplicon diluted 1:50 in ddH2O, 9.5 μl HiDi formamide (ABI) and 0.5 μl 500 LIZ size standard was prepared and DNA products were electrophoresed on an ABI 3130xl automated sequencer. Data was analysed using ABI GeneMapper software v4.0 (Applied Biosystems (ABI)).

### Southern Blotting

Genomic DNA (gDNA) was concentrated for restriction endonuclease digestion using CA clean (Microzone) according to the manufacturer's instructions. A total of 3-10 ug of gDNA was digested overnight with AluI (20 u) and DdeI (20 u) in Restriction Buffer 2 (New England Biolabs) at 37° C. prior to electrophoresis for 18 hours at 1.5 volts/cm in 0.8% agarose containing 0.5×TBE. DNA was transferred to positively charged nylon membrane (Roche Applied Science) by capillary blotting and baked at 80° C. for 2 hours. The hybridisation probe was an oligonucleotide from Eurofins MWG Operon (Germany) and comprised five hexanucleotide repeats (GGGGCC)₅ (SEQ ID NO: 1) labelled 3′ and 5′ with digoxigenin (DIG). Filter hybridisation was undertaken in a Hybaid Oven as recommended in the DIG Application Manual (Roche Applied Science) except for the supplementation of DIG Easy Hyb buffer with 100 ug/ml denatured fragmented salmon sperm DNA. Following prehybridisation in 30 ml DIG Easy Hyb buffer at 48° C. for 4 hours hybridisation was allowed to proceed at 48° C. overnight in fresh pre-heated DIG Easy Hyb buffer containing the probe. A total of 1 ng of labelled oligonucleotide probe was used per ml of hybridisation solution. Membranes were then subjected to 50 ml washes in the hybridisation bottle. Initially in 2× standard sodium citrate (SSC), 0.1% sodium dodecyl sulphate (SDS), ramping the oven from 48° C. to 65° C. followed by fresh solution at 65° C. for 15 minutes and then further 15 minute washes in 0.5×SSC, 0.1% SDS and 0.2×SSC, 0.1% SDS at 65° C. Detection of the hybridised probe DNA was carried out as recommended in the DIG Application Manual using CSPD ready-to-use (Roche Applied Science) as chemiluminescent substrate. Signals were visualised on Fluorescent Detection Film (Roche Applied Science) after 1 to 5 hours. All samples were electrophoresed against DIG labelled DNA molecular weight markers II and VII (Roche Applied Science). Hexanucleotide repeat number was estimated by interpolation using a plot of log₁₀ base pair number against migration distance which was created in Excel (Microsoft). Maximum, minimum and modal size, were recorded for each patient with expanded repeats. No signal from the pathogenic range was observed using this method in 50 rpPCR negative control samples.

### Results

2974 patient samples comprised 6 disease cohorts (FTLD, AD, MND, sCJD, HD-like, or other neurodegenerative diseases). The purpose of the extended patient screen was to characterise the phenotypic range and provide varied case samples for subsequent genotype-phenotype correlation. The number of rpPCR patient samples estimated to have >30 repeats were 28/375 FTLD (7.5%), 11/904 AD (1.2%), 29/360 MND (8.1%), 1/470 sCJD (0.2%), 9/444 other neurodegenerative diseases (2.0%), 7/421 HD-like (1.7%). In total 85 C9orf72 expansion samples (2 samples were identified retrospectively to be in both the HD-like and FTD cohorts and were removed from the former category). 18 FTLD cases from the UCL FTLD DNA cohort have been described in detail elsewhere, but are included here for comparison purposes (Mahoney et al., 2012). Mean age at onset was 54.6 years and did not differ between cohorts; autosomal dominant pattern inheritance of early onset neurodegenerative disease (at least one other relative) was documented in 29%. Notable atypical clinical presentations/clinical features included psychiatric symptoms (treatment with major tranquilisers in at least three), movement disorders (Parkinsonism in two, several in the HD-like cohort with chorea, myoclonus prompting consideration of CJD in one). On case note review from the AD series where more details were available, C9orf72 cases had overlap clinical features of FTD; there were no autopsy findings available.

Combining the UK control cohorts 12/7599 (1 in 632, 95% CI 0.08-0.28%) C9ORF72 expansions were found. Notably, individuals from the 58BC are now 54 years old; on retrospective case review, one of these individuals had already died with a clinical diagnosis of MND and was subsequently moved to the MND cohort. Excluding this individual, the control prevalence was 11/7598 (1 in 691 or 0.15%, 95% CI 0.07-0.26%).

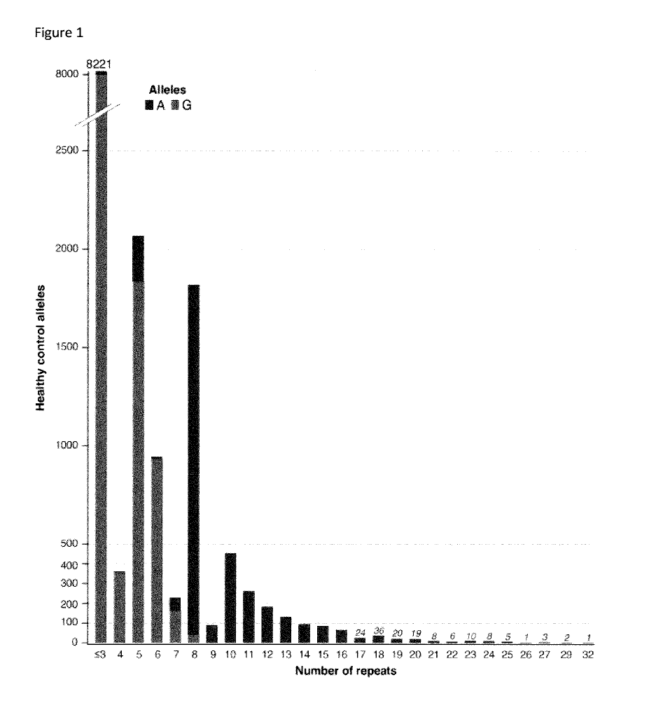

We went on to look at the stability of the C9orf72 hexanucleotide repeat region in the entire CEPH family series (table 1, 2 supplementary). No large expansions (>30 repeats) were found. Three changes were seen in repeat size between generations, with no maternal transmission (11→12, 21→22, 22→20) giving an overall intergenerational repeat change rate of 0.29%. All changes were verified by repeat rpPCR and fluorescent labelled PCR size fractionation. Haplotypes were confirmed by analysis of linked SNPs and microsatellites (FIG. 1 supplementary). All intergenerational changes occurred on an rs3849942A haplotype background and all occurred from a starting repeat length >10 (P=0.001, MWU test). The largest repeat in the CEPH families (22 repeats) changed size twice in the same family; 21→22 paternal grandparent (142311) to father (142301) and 22→20 from father to son (142307) (table 2 supplementary). These data support the inference that larger expansions and/or the rs3849942A haplotype background (the “risk” haplotype) are associated with considerable instability of repeat length.

As reported by others, we found strong linkage disequilibrium (LD) between repeat length and neighbouring SNPs (see FIG. 1 for rs3849942; Majounie et al., 2012). 5400 WTCCC2 healthy control individuals were assessed using fastPHASE, generating haplotypes across the chromosome 9 region 27471905-27562634 (˜91 Kb). Of 10,400 haplotypes, 2597 (25%) were rs3849942A, 16 of which were described by Mok et al. as part of the susceptibility haplotype associated with C9 expansion and disease. Of the 2597 rs3849942A haplotpyes we detected, 2435 (94%) were identical to each other and the disease related. The disease associated SNP haplotype is therefore common in the healthy UK population, the outstanding question was therefore whether all cases share an ancient single common ancestor, or whether this haplotype confers increased risk of mutation, many of these having occurred in human history.

We sought to distinguish these possibilities by testing for evidence of a founder effect by looking at 10 microsatellites over the surrounding 13.1 MB (two microsatellites were within 300 kb of C9orf72) to provide evidence of shared ancestry beyond the SNP haplotype. At the time an expansion mutation occurs it is linked with all microsatellite variation on the same chromosome; over time however, this mutation associated haplotype will break down due to both recombination occurring between C9orf72 and the microsatellite, and alteration of microsatellite repeat length by mutation. We found 8 different microsatellite alleles linked to C9orf72 expansions at 2 microsatellites within 300 kb with an estimated recombination rate with C9orf72 of less than once in 200 generations (Kong et al., 2002). We empirically estimated the total number of possible microsatellite haplotypes in a subset of 48 expansion cases. We found at least 60 different haplotypes based on incompatibility of genotypes. Using the same empirical methods we made similar estimates in 48 CEPH parents and predicted at least 76 haplotypes (not statistically significantly different from cases). Haplotyping using genotypes from children of the same CEPH parents revealed that all 96 haplotypes were unique, implying that all or a very high proportion of haplotypes in the case series were also unique. The microsatellite allele frequencies associated with C9orf72 expansions as a group were indistinguishable from controls including those linked with rs3849942A (table 2 supplementary data). These data provide strong evidence against shared ancestry of a large proportion of C9orf72 expansion patients from the UK.

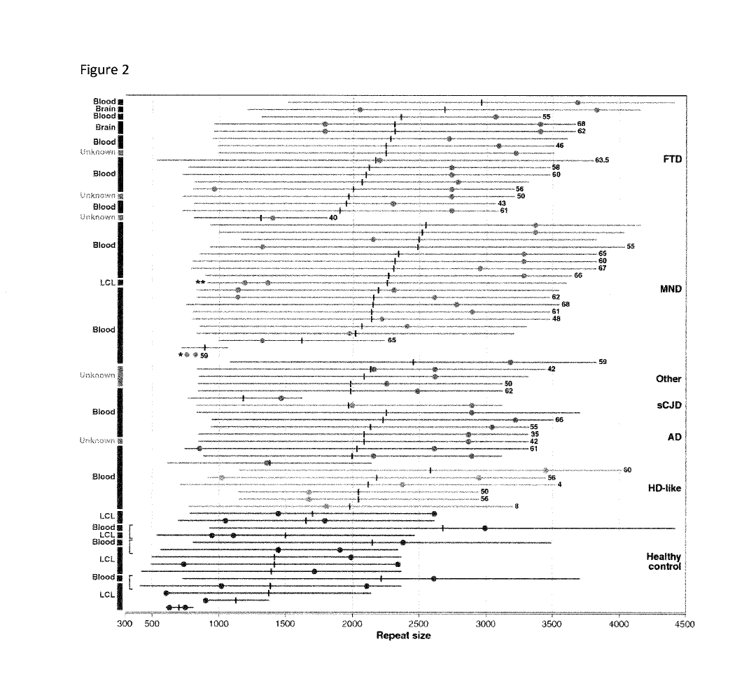

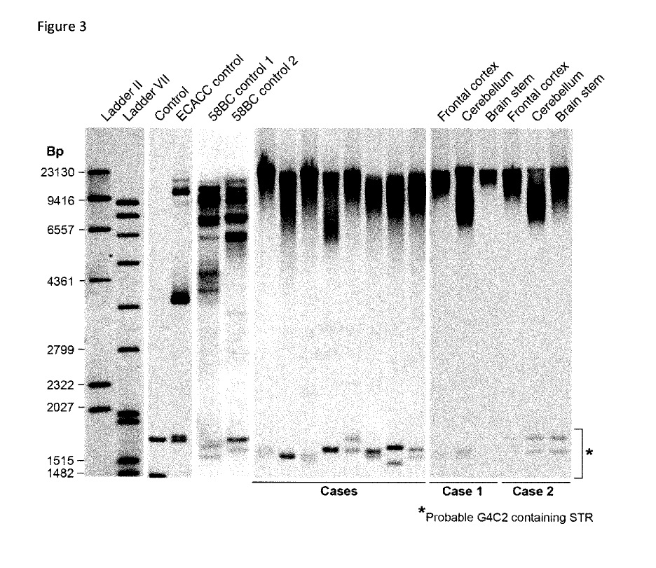

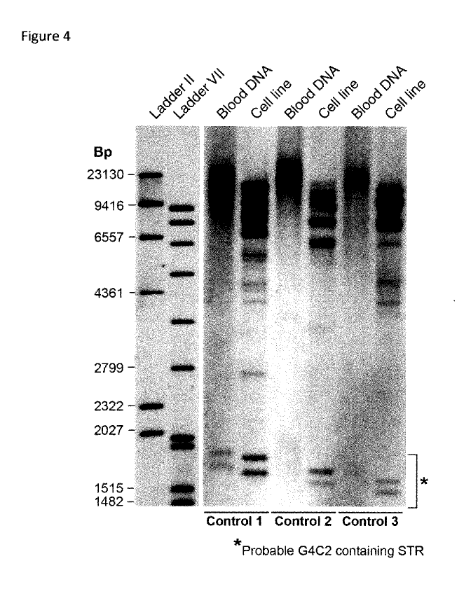

We modified the Southern blotting method of DeJesus-Hernandez et al. with the aim of enhancing the expansion signal when using modest amounts of genomic DNA (see methods, FIG. 2, 3, 4). A more sensitive blotting methodology would allow direct estimates of expansion size in a large and more representative sample series and allow genotype-phenotype correlation. This was done by using a more complete restriction endonuclease digestion of genomic DNA and a (GGGGCC)₅ (SEQ ID NO: 1) DIG probe rather than one specific to adjacent DNA sequence. We found no large expansions in 50 rpPCR negative samples, and confirmed large expansions in 68/69 rpPCR positive samples, demonstrating the high specificity of the modified protocol. Observed patterns were remarkably variable, with long smears interrupted by one or more modal points (see FIGS. 2-4 for individuals estimates of repeat size). For statistical analysis we compared multiple estimates of repeat size based on smear maxima (range 790-4400) and minima (400-1500), midpoint of smear (700-3000), and mode (630-3800) or modal points (630-2200, 20 samples with more than one mode). Lymphocyte cell line DNA was associated with smaller repeats sizes and a distinct multi-modal banding pattern (FIG. 3, 4), which we have assumed relates to the pauciclonal origins of DNA in cell lines. Surprisingly, all rpPCR positive control cohort samples had large expansions (>400 repeat smear minima) and overlap the range seen in cases. Three control samples were available from blood and all were typical of cases (FIG. 4).

Minima, maxima, midpoint and modal estimates of repeat size were all statistically significantly correlated with some aspects of clinical phenotype, however importantly, there were no differences between any two disease cohorts by any repeat size measure (P>0.1 all pairwise comparisons, Tukey post hoc test, ANOVA). Cell line repeat sizes (largely controls) were smaller than blood extracted DNA by all measures (ANOVA, post hoc Tukey test P<0.01). The modal point of repeat size correlated with age at clinical onset (increasing age, increasing repeat size, Pearson correlation 0.38, P=0.02) however other repeat size metrics did not significantly correlate. The presence of a family history was associated with smaller repeat sizes measured by all metrics (e.g. modal size, t-test, P=0.003). In two cases we blotted DNA extracted from frontal cortex, brain stem and cerebellum and observed marked differences between different brain regions (FIG. 3), although more samples will need to be analysed for consistency and a statistical analysis.

### Discussion

We have screened a large case and control series and developed a new Southern blotting methodology to understand the prevalence of the C9orf72 expansion, its pathogenicity and extend genotype-phenotype correlations. Whereas earlier studies suggested a healthy control upper limit of 30 repeats, we found that large expansions (>400 repeats) in C9orf72 are not infrequent in the UK population at a rate of around 1 in 600. This is considerably more prevalent than would be expected from epidemiological studies. Surprisingly, control individual expansions extracted from blood are indistinguishable from case samples. Despite considerable heterogeneity and evidence of somatic mutation expansion metrics did not differ in diagnostic categories. Finally, we provide evidence in support of the risk haplotype hypothesis of mutation origin.

In order to approximate the size of pathogenic expansions we developed a Southern blot methodology that utilised a DIG labelled oligonucleotide probe comprising 5 hexanucleotide repeats (GGGGCC)₅ (SEQ ID NO: 1). Our concept was that this probe with multiple hybridisation sites within the repeat expansion would give a stronger signal than a single copy probe hybridising to the restriction fragment containing the repeats. The choice of two frequently cutting restriction endonucleases with restriction sites that closely flank the repeat region produced highly fragmented gDNA (˜200-300 bp modal size). This allowed the oligonucleotide repeat probe to have hybridisation specificity for the C9orf72 expansion. No hybridisation signal was detected for restriction fragments above 1700 base pairs in 50 controls allowing for unambiguous and sensitive detection of all C9orf72 expansions greater than ˜275 repeats.

The refined methodology allows for sizing of as little as 3 μg of gDNA. It also allows for a more accurate definition of the range which is observed in gDNA samples extracted from tissue and which most probably results from somatic mutation. In lymphoblastoid cell line DNA from controls carrying large expansions the method detects multiple bands of variable intensity highlighting the degree of pauciclonality that exists in such lines. It has been previously reported that some DNA fragments containing repeats have abnormal migration in agarose compared with more typical gDNA fragments and that the amount of flanking sequence in the fragment containing the expansion may also have an influence(Mahoney et al., 2012). Therefore overall repeat number could potentially appear different with the use of a different Southern methodology. We would therefore emphasise relative size of expansions rather than exact number of repeats. It also remains a possibility that determination of maximum repeat number could be restricted by the modal size of undigested gDNA.

The prevalence of large expansions in the healthy UK population is intriguing. Lifetime risk of MND has been estimated as ˜1 in 430. Lifetime risk of FTD is less well understood, but incidence measured in two studies was 3.5 and 4.1 per 100,000 in the 45-64 age cohort, comparable to MND, implying a similar lifetime risk. Using C9orf72 mutation frequencies based on a recent large study and estimates of the proportion of MND and FTD with familial disease (Majounie et al., 2012; Hanby et al., 2011; Rohrer et al., 2009), the lifetime risk of C9orf72 associated FTD or MND is approximately 1 in 2000. Whilst the uncertainties in the true lifetime risk of FTD prevent a formal statistical comparison with the frequency of C9orf72 expansions, the estimate differs considerably from the 1 in 631 central estimate of our population genetic study. There are several potential explanations for this discrepancy: first, the lifetime risk of FTD may in fact be much greater than MND; second, many clinical syndromes caused by C9orf72 expansions are not diagnosed as FTD or MND; and third, the penetrance of the expansion is much lower than predicted by family studies of currently ascertained cases. Our case screen supports the second suggestion as C9ORF72 expansions were found in all neurodegenerative disease categories we tested and a third of our case series had diagnoses other than FTD and/or MND. Several of these diseases (notably AD) are highly prevalent conditions in old age populations, which may therefore harbour large numbers of C9orf72 cases. These data emphasise the potential importance of the C9orf72 expansion mutation in neurodegeneration with our estimates suggesting there may be approximately 90,000 mutation carriers in the UK.

Although the presence and size of C9orf72 expansions did not differ between diagnostic groups, we did identify a correlation between the age of onset and expansion size. From three brain regions in two cases, we also found evidence of marked and consistent differences within an individual, indicating considerable scope for heterogeneity in specific cell types; large smears and variable patterns were seen in blood extracted DNA. These findings are likely to be due to somatic instability. Variation in expansion size between brain regions and with age is consistent with studies of GAA-repeat expansion size in Friedreich's ataxia, which again implicate somatic mutation. This may be one explanation for the phenotypic heterogeneity and incomplete penetrance of C9orf72 expansion diseases.

The considerable instability of the expansion suggested by somatic mutation raises questions about the founder hypothesis of mutation origin. This proposes that a large proportion of cases share a common ancestor with a single mutational event (Majounie et al., 2012). We used genotyping of the surrogate marker rs3849942 for the haplotype at risk of expansion to make inferences about the stability and origin of the expansion in UK population history. In keeping with previous reports we found a distinct difference in the distribution pattern of repeat numbers in controls for alleles of the “risk” haplotype as compared with other haplotypes, with longer repeats linked to the “risk” haplotype (Dejesus-Hernandez et al., 2012). Additionally, all 11 control samples with expansions greater than 400 repeats were either heterozygous or homozygous for the “risk” haplotype. In the CEPH pedigrees we found 3 alterations of repeat size between generations, which all occurred on the “risk” haplotype, further indicating that the repeat region on this haplotype is less stable. In addition, using microsatellite analysis we found no evidence of a founder effect, with no evidence of shared haplotypes beyond the SNP “risk” haplotype found in controls. Two of the microsatellites genotyped were within 300 kb of C9orf72 and would be expected to show residual linkage disequilibrium (LD) if a single mutational event in Finland resulted in a large proportion of UK cases. Whilst there is little doubt a founder effect has resulted in the high prevalence in Finland, taken together, our data are more compatible with the “risk” haplotype hypothesis, linked to larger-normal-range (>6 repeats) and more unstable repeats, consequently generating very large expansions in unrelated individuals many times throughout human history and explaining the prevalence of mutations in countries distant from Finland.

In summary we have developed a reliable method to approximate the C9orf72 expansion size which may have clinical diagnostic utility. Our data emphasise the importance of this mutation in neurodegeneration and common neurodegenerative diseases outside of the FTD/MND spectrum, and provide direct evidence for repeat instability, somatic mutation, and the “risk” haplotype hypothesis of mutation origin.

### REFERENCES

All documents mentioned in this specification are incorporated herein by reference in their entirety.
1. Renton A E, Majounie E, Waite A, et al. A Hexanucleotide Repeat Expansion in C9ORF72 Is the Cause of Chromosome 9p21-Linked ALS-FTD. Neuron 2011; 72: 257-68.
2. Dejesus-Hernandez M, Mackenzie I R, Boeve B F, et al. Expanded GGGGCC Hexanucleotide Repeat in Noncoding Region of C9ORF72 Causes Chromosome 9p-Linked FTD and ALS. Neuron 2011; 72: 245-56.
3. Mahoney C J, Beck J, Rohrer J D, et al. Frontotemporal dementia with the C9ORF72 hexanucleotide repeat expansion: clinical, neuroanatomical and neuropathogenic features. Brain 2012; 135: 736-50.
4. Majounie E, Renton A E, Mok K, et al. Frequency of the C9orf72 hexanucleotide repeat expansion in patients with amyotrophic lateral sclerosis and frontotemporal dementia: a cross-sectional study. Lancet Neurology 2012; 11: 323-30.
5. Kong A, Gudbjartsson D F, Sainz J, et al. A high-resolution recombination map of the human genome. Nature Genetics 2002; 31: 241-7.
6. Hanby M F, Scott K M, Scotton W, et al. The risk to relatives of patients with sporadic amyotrophic lateral sclerosis. Brain 2011; 134: 3451-4.
7. Rohrer J D, Guerreiro R, Vandrovcova J, et al. The heritability and genetics of frontotemporal lobar degeneration. Neurology 2009; 73: 1451-6.

1. A method of estimating the size of a disease-associated polynucleotide repeat expansion in a gene, the method comprising:
(a) contacting the sample of genomic DNA from an individual with one or more restriction enzymes, wherein the restriction enzymes have restriction sites flanking the region of genomic DNA containing the polynucleotide repeat expansion and are capable of cutting the genomic DNA outside of the fragment containing the polynucleotide repeat expansion into a plurality of DNA fragments;
(b) optionally separating the nucleic acid fragment containing the polynucleotide repeat expansion from the plurality of DNA fragments;
(c) contacting the nucleic acid fragment containing the polynucleotide repeat expansion with a hybridisation probe capable of targeting multiple sites within the polynucleotide repeat expansion; and
(d) detecting the hybridisation of the hybridisation probe to the polynucleotide repeat expansion to estimate the size of the disease-associated polynucleotide repeat expansion;
wherein the one or more restriction enzymes do not cut within the repeat expansion and are frequent cutting restriction enzymes capable of cutting genomic DNA into fragments of a modal size below the size of the repeat expansion,
and wherein the disease associated with the polynucleotide repeat expansion is a neurological disease.

2. The method of claim 1, wherein the restriction enzymes are capable of cutting the genomic DNA into fragments of a modal size no greater than 300 base pairs in length.

3. The method of claim 1, wherein the sample of genomic DNA is contacted with more than one restriction enzyme.

4. The method of claim 1, wherein restriction sites flanking the region of genomic DNA containing the polynucleotide repeat expansion are within a distance (in base pairs) less than the modal size of the fragmented DNA from the 3′ and/or 5′ ends of the polynucleotide repeat sequence.

5. The method of claim 1, wherein the restriction enzymes are AluI and DdeI.

6. The method of claim 1, wherein the hybridisation probe comprises a multimeric sequence capable of hybridising to at least one tandem repeat of a polynucleotide sequence.

7. The method of claim 6, wherein the tandem repeat of a polynucleotide sequence is comprised in a polynucleotide repeat expansion.

8. The method of claim 6, wherein the hybridisation probe comprises n number of repeats of a sequence capable of hybridising to the polynucleotide repeat expansion, where n is between 2 and 10.

9. The method of claim 6, wherein the hybridisation probe comprises a multimeric sequence of a polynucleotide sequence as defined in Table 1, or a complementary sequence thereof.

10. The method of claim 6, wherein the hybridisation probe comprises a label for detection.

11. The method of claim 10, wherein the label is a fluorescent, chemiluminescent, chromogenic, enzymatic, radioactive or hapten label.

12. The method of claim 11, wherein the label is a digoxigenin (DIG).

13. The method of claim 1, wherein the polynucleotide repeat expansion comprises 20 repeats or more.

14. The method of claim 1, wherein the polynucleotide repeat expansion comprises 50 repeats or more.

15. The method of claim 1, wherein the polynucleotide repeat expansion comprises 100 repeats or more.

16. The method of claim 1, wherein the polynucleotide repeat expansion is at least 1650 base pairs in length.

17. The method of claim 1, wherein the size of the polynucleotide repeat expansion is estimated by reference to one or more DNA fragments of a known size.

18. The method of claim 1, wherein the size of polynucleotide repeat expansion is variable in a sample from an individual.

19. The method of claim 18, wherein the method comprises an additional step of determining the range of variation in the size of polynucleotide repeat expansion.

20. The method of claim 1, further comprising the initial step of obtaining a sample of genomic DNA from an individual.

21. The method of claim 1, wherein the method does not amplify the sample of genomic DNA.

22. The method of claim 1, wherein the method is capable of estimating the size of a polynucleotide repeat expansion in a genomic DNA sample of 5 ug or less.

23. The method of claim 1, wherein separating the nucleic acid fragments containing the polynucleotide repeat expansion from the plurality DNA fragments of a modal size below the size of the expansion length is achieved by resolving the sample resulting from step (c) by electrophoresis.

24. The method of claim 1, further comprising the step of:
correlating the estimated size of the polynucleotide repeat expansion with the range of sizes considered to be non-pathogenic or pathogenic for the disease, wherein an estimated size within the range considered to be pathogenic is indicative of disease.

25. The method of claim 24, wherein a disease is indicated by the detection of an expansion estimated to be within the range of pathogenic expansion sizes for the disease shown in Table 1.

26. The method of claim 1, further comprising the step of:
correlating the estimated size of the polynucleotide repeat expansion with the range of sizes considered to be non-pathogenic or pathogenic for the disease, wherein an estimated size between these two ranges or in the upper 10% of expansion sizes in the non-pathogenic range is indicative of a predisposition of offspring of the individual to the disease.

27. The method of claim 26, wherein a predisposition to a disease associated with polynucleotide repeat expansion is indicated by the detection of an expansion estimated to be within the upper 10% of the range of non-pathogenic expansion sizes, or in between ranges for normal and pathogenic expansion sizes for the disease shown in Table 1.

28. The method of claim 1, further comprising the step of:
correlating the estimated size of the polynucleotide repeat expansion with the range of sizes associated with a particular age of onset for the disease.

29. The method of claim 28, wherein a larger repeat expansion size within the pathogenic range is indicative of an earlier age of onset for the disease.

30. The method of claim 1, further comprising the step of:
correlating the estimated size of the polynucleotide repeat expansion with the range of sizes associated with a particular clinical phenotype of a disease.

31. The method of claim 1, further comprising the step of:
correlating the estimated size of the polynucleotide repeat expansion with the range of sizes associated with a particular disease prognosis.

32. The method of claim 31, wherein a larger repeat expansion size within the pathogenic range is indicative of a poorer disease prognosis.

33. The method of claim 1, further comprising the step of:
correlating the estimated size of the polynucleotide repeat expansion with the range of sizes associated with a particular response to treatment for a disease.

34. The method of claim 1, comprising an additional step of determining the actual size of the polynucleotide repeat expansion.

35. The method of claim 1, wherein the genomic DNA sample is isolated from an individual in which a polynucleotide repeat expansion is already known.

36. The method of claim 35, wherein the polynucleotide repeat expansion already known was detected by PCR, DNA sequencing, rpPCR or conventional Southern blotting.

37. The method of claim 36, wherein the polynucleotide repeat expansion already known was detected by rpPCR.

38. The method of claim 1, wherein the disease associated with the polynucleotide repeat expansion is a neurodegenerative disease.

39. The method of claim 38, wherein the disease is frontotemporal dementia (FTD), amyotrophic lateral sclerosis (ALS), motor neuron disease (MND), Alzheimer's disease (AD), Huntington's disease (HD), Friedreich's ataxia (FRDA), X-linked spinal and bulbar muscular atrophy (SBMA), fragile X syndrome (FRAXA), fragile X associated tremor/ataxia syndrome (FXTAS), fragile XE mental retardation (FRAXE), myotonic dystrophy (DM), spinocerebellar ataxias (SCAs), corticobasal syndrome (CBS), ataxic syndrome and dentatorubal-pallidoluysian atrophy (DRPLA).

40. The method of claim 1, wherein the polynucleotide repeat expansion is in the C9orf72 gene.

41. The method of claim 40, wherein the hybridisation probe comprises the sequence (GGGGCC)n (SEQ ID NO: 2) or (CCCCGG)n (SEQ ID NO: 3), where n is between 2 and 10.

42. A kit for estimating the size of a disease-associated polynucleotide repeat expansion in a gene, the kit comprising:
one or more restriction enzymes, wherein the restriction enzymes have restriction sites flanking the region of genomic DNA containing the polynucleotide repeat expansion and which are capable of cutting the genomic DNA outside of the polynucleotide repeat expansion into a plurality of small DNA fragments;
a hybridisation probe capable of targeting multiple sites within the polynucleotide repeat expansion; and
wherein detecting hybridisation of the hybridisation probe to the polynucleotide repeat expansion enables the size of the disease-associated polynucleotide repeat expansion to be estimated.


SEQUENCE LISTING (28 sequences)

SEQ ID NO: 1 - DNA, 30 bp, Artificial sequence
  Synthetic hybridisation probe
ggggccggggccggggccggggccggggcc

SEQ ID NO: 2 - DNA, 60 bp, Artificial sequence
  Synthetic hybridisation probe
ggggccggggccggggccggggccggggccggggccggggccggggccggggccggggcc

SEQ ID NO: 3 - DNA, 60 bp, Artificial sequence
  Synthetic hybridisation probe
ccccggccccggccccggccccggccccggccccggccccggccccggccccggccccgg

SEQ ID NO: 4 - DNA, 105 bp, Homo sapiens
  misc_feature (1)..(105): Non-pathogenic repeat size expansion of motif CAG within the DRPLA gene. Up to 29 CAG motifs can either be present or absent - represents a range of 6 - 35 CAG motifs
cagcagcagcagcagcagcagcagcagcagcagcagcagcagcagcagcagcagcagcagcagcagcagcagcagcagcagcagcagcagcagcagcagcagcag

SEQ ID NO: 5 - DNA, 108 bp, Homo sapiens
  misc_feature (1)..(108): Non-pathogenic repeat size expansion of motif CAG within the AR gene. Up to 27 CAG motifs can either be present or absent - represents a range of 9 - 36 CAG motifs
cagcagcagcagcagcagcagcagcagcagcagcagcagcagcagcagcagcagcagcagcagcagcagcagcagcagcagcagcagcagcagcagcagcagcagcag

SEQ ID NO: 6 - DNA, 96 bp, Homo sapiens
  misc_feature (1)..(96): Non-pathogenic repeat size expansion of motif CAG within the ATXN2 gene. Up to 18 CAG motifs can either be present or absent - represents a range of 14 - 32 CAG motifs
cagcagcagcagcagcagcagcagcagcagcagcagcagcagcagcagcagcagcagcagcagcagcagcagcagcagcagcagcagcagcagcag

SEQ ID NO: 7 - DNA, 120 bp, Homo sapiens
  misc_feature (1)..(120): Non-pathogenic repeat size expansion of motif CAG within the ATXN3 gene. Up to 28 CAG motifs can either be present or absent - represents a range of 12 - 40 CAG motifs
cagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 8 - DNA, 54 bp, Homo sapiens
  misc_feature (1)..(54): Non-pathogenic repeat size expansion of motif CAG within the CACNA1A gene. Up to CAG motifs can either be present or absent - represents a range of 4 - 18 CAG motifs
cagcagcagcagcagcagcagcagcagcagcagcagcagcagcagcagcagcag

SEQ ID NO: 9 - DNA, 51 bp, Homo sapiens
  misc_feature (1)..(51): Non-pathogenic repeat size expansion of motif CAG within the ATXN7 gene. Up to 10 CAG motifs can either be present or absent - represents a range of 7 - 17 CAG motifs
cagcagcagcagcagcagcagcagcagcagcagcagcagcagcagcagcag

SEQ ID NO: 10 - DNA, 111 bp, Homo sapiens
  misc_feature (1)..(111): Non-pathogenic repeat size expansion of motif CTG within the SCA8 gene. Up to 21 CTG motifs can either be present or absent - represents a range of 16 - 37 CTG motifs
ctgctgctgctgctgctgctgctgctgctgctgctgctgctgctgctgctgctgctgctgctgctgctgctgctgctgctgctgctgctgctgctgctgctgctgctgctg

SEQ ID NO: 11 - DNA, 126 bp, Homo sapiens
  misc_feature (1)..(126): Non-pathogenic repeat size expansion of motif CAG within the TBP gene. Up to 17 CAG motifs can either be present or absent - represents a range of 25 - 42 CAG motifs
cagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 12 - DNA, 159 bp, Homo sapiens
  misc_feature (1)..(159): Non-pathogenic repeat size expansion of motif CGG within the FMR1 gene. Up to 47 CGG motifs can either be present or absent - represents a range of 6 - 53 CGG motifs
cggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcgg

SEQ ID NO: 13 - DNA, 105 bp, Homo sapiens
  misc_feature (1)..(105): Non-pathogenic repeat size expansion of motif GCC within the FMR2 gene. Up to 29 GCC motifs can either be present or absent - represents a range of 6 - 35 GCC motifs
gccgccgccgccgccgccgccgccgccgccgccgccgccgccgccgccgccgccgccgccgccgccgccgccgccgccgccgccgccgccgccgccgccgccgcc

SEQ ID NO: 14 - DNA, 102 bp, Homo sapiens
  misc_feature (1)..(102): Non-pathogenic repeat size expansion of motif GAA within the FXN gene. Up to 27 GAA motifs can either be present or absent - represents a range of 7 - 34 GAA motifs
gaagaagaagaagaagaagaagaagaagaagaagaagaagaagaagaagaagaagaagaagaagaagaagaagaagaagaagaagaagaagaagaagaagaa

SEQ ID NO: 15 - DNA, 264 bp, Homo sapiens
  misc_feature (1)..(264): Pathogenic repeat size expansion of motif CAG within the DRPLA gene. Up to 39 CAG motifs can either be present or absent - represents a range of 49 - 88 CAG motifs
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 16 - DNA, 108 bp, Homo sapiens; identical to SEQ ID NO: 5
  misc_feature (1)..(108): Pathogenic repeat size expansion of motif CAG within the HTT gene. Represents >35 CAG motifs

SEQ ID NO: 17 - DNA, 186 bp, Homo sapiens
  misc_feature (1)..(186): Pathogenic repeat size expansion of motif CAG within the AR gene. Up to 24 CAG motifs can either be present or absent - represents a range of 38 - 62 CAG motifs
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 18 - DNA, 231 bp, Homo sapiens
  misc_feature (1)..(231): Pathogenic repeat size expansion of motif CAG within the ATXN2 gene. Up to 44 CAG motifs can either be present or absent - represents a range of 33 - 77 CAG motifs
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 19 - DNA, 258 bp, Homo sapiens
  misc_feature (1)..(258): Pathogenic repeat size expansion of motif CAG within the ATXN3 gene. Up to 31 CAG motifs can either be present or absent - represents a range of 55 - 86 CAG motifs
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 20 - DNA, 90 bp, Homo sapiens
  misc_feature (1)..(90): Pathogenic repeat size expansion of motif CAG within the CACNA1A gene. Up to 9 CAG motifs can either be present or absent - represents a range of 21 - 30 CAG motifs
cagcagcagcagcagcagcagcagcagcagcagcagcagcagcagcagcagcagcagcagcagcagcagcagcagcagcagcagcagcag

SEQ ID NO: 21 - DNA, 360 bp, Homo sapiens
  misc_feature (1)..(360): Pathogenic repeat size expansion of motif CAG within the ATXN7 gene. Up to 82 CAG motifs can either be present or absent - represents a range of 38 - 120 CAG motifs
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 22 - DNA, 750 bp, Homo sapiens
  misc_feature (1)..(750): Pathogenic repeat size expansion of motif CTG within the SCA8 gene. Up to 140 CTG motifs can either be present or absent - represents a range of 110 - 250 CTG motifs
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 23 - DNA, 189 bp, Homo sapiens
  misc_feature (1)..(189): Pathogenic repeat size expansion of motif CAG within the TBP gene. Up to 16 CAG motifs can either be present or absent - represents a range of 47 - 63 CAG motifs
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 24 - DNA, 693 bp, Homo sapiens
  misc_feature (1)..(693): Pathogenic (Fragile X syndrome) repeat size expansion of motif CGG within the FMR1 gene. Represents >230 CGG motifs
cggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcgg

SEQ ID NO: 25 - DNA, 600 bp, Homo sapiens
  misc_feature (1)..(600): Pathogenic (Fragile X associated tremor/ ataxia syndrome) repeat size expansion of motif CGG within the FMR1 gene. Up to 145 CGG motifs can either be present or absent - represents a range of 55 - 200 CGG motifs
cggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcgg

SEQ ID NO: 26 - DNA, 603 bp, Homo sapiens
  misc_feature (1)..(603): Pathogenic repeat size expansion of motif GCC within the FMR2 gene. Represents >200 GCC motifs
gccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgcc

SEQ ID NO: 27 - DNA, 303 bp, Homo sapiens
  misc_feature (1)..(303): Pathogenic repeat size expansion of motif GAA within the FXN gene. Represents >100 GAA motifs
gaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaa

SEQ ID NO: 28 - DNA, 153 bp, Homo sapiens
  misc_feature (1)..(153): Pathogenic repeat size expansion of motif CTG within the DMPK gene. Represents >50 CTG motifs
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg